(19) United States
(12) Patent Application Publication
(10) Pub. No.: US 2007/0238109 A1
(43) Pub. Date: Oct. 11, 2007

(54) METHOD AND APPARATUS FOR PURIFYING NUCLEIC ACID ON HYDROPHILIC SURFACE OF SOLID SUPPORT USING HYDROGEN BONDING

(75) Inventors: Jun-hong MIN, Yongin-si (KR); In-ho LEE, Yongin-si (KR); Chang-eun YOO, Seoul (KR); Ki-woong HAN, Seoul (KR); Young-rok KIM, Yongin-si (KR)

Correspondence Address: CANTOR COLBURN, LLP, 55 GRIFFIN ROAD SOUTH, BLOOMFIELD, CT, 06002, US

(73) Assignee: SAMSUNG ELECTRONICS CO., LTD., Suwon-si (KR)

(21) Appl. No.: 11/553,657

(22) Filed: Oct. 27, 2006

(30) Foreign Application Priority Data
Apr. 6, 2006 (KR) 10-2006-0031490

(51) Int. Cl. C12Q 1/68; C12M 3/00

(52) U.S. Cl. 435/6; 435/287.2

(57) ABSTRACT

Provided is a method of purifying nucleic acid, the method including: contacting a nucleic acid-containing sample and a solution containing a kosmotropic salt on a solid support having a hydrophilic functional group on its surface to bind the nucleic acid to the solid support. Since the solid support is used as it is without any surface treatment, manufacture of the apparatus is very easy, and nucleic acid can be bound to the solid support without specific additives in a wide pH range, so that the apparatus can be used for a Lab-On-a-Chip.

This application claims priority to Korean Patent Application No. 10-2006-0031490, filed on Apr. 6, 2006, and all the benefits accruing therefrom 35 U.S.C. §119(a), the contents of which are herein incorporated by reference in its entirety.

### BACKGROUND OF THE INVENTION

1. Field of the Invention

The present invention relates to a method and apparatus for purifying nucleic acids on a hydrophilic surface of a solid support using hydrogen bonding.

2. Description of the Related Art

Methods of isolating DNA from cells are performed using materials that have a proclivity for binding to DNA. Examples of materials used for isolating DNA include silica, glass fiber, anion exchange resin and magnetic beads (Rudi, K. et al., Bio Techniques 22, 506-511 (1997); and Deggerdal, A. et al., Bio Techniques 22, 554-557 (1997)). To avoid manual operation and to remove operator error, automated machines have been developed for high-throughput DNA extraction.

The production of high purity double-stranded plasmid DNAs, single-stranded phage DNAs, chromosomal DNAs, and agarose gel-purified DNA fragments is very important in molecular biology. Ideal methods of purifying DNAs should be simple and quick, and include little additional manipulation of samples. The DNAs obtained using such methods can be used for direct transformation, restriction enzyme analysis, ligation, or sequencing. Such methods are very attractive in the automated production of DNA samples, which is favored in research and diagnosis labs.

Conventionally, a method of purifying nucleic acid using a solid phase is known. For example, U.S. Pat. No. 5,234,809 discloses a method of purifying nucleic acid using a solid phase to which nucleic acids are bound, the method including: mixing a starting material, a chaotropic (i.e., water-disrupting) material, and a nucleic acid binding solid phase; separating the solid phase with the nucleic acid bound thereto from the liquid, and washing the solid phase nucleic acid complexes. However, this method is time consuming and complicated, and thus is not suitable for a Lab-On-a-Chip (“LOC”). The method also has a problem in that the chaotropic material should be used.

U.S. Pat. No. 6,291,166 discloses a method of archiving nucleic acid using a solid-phase matrix. This method is advantageous in that since nucleic acids are irreversibly bound to the solid-phase matrix, delayed analysis or repeated analysis of the nucleic acid solid-phase matrix complexes is possible. However, according to this method, alumina, which has a positively charged surface, needs to be rendered hydrophilic by addition of basic materials, such as NaOH. Nucleic acids are irreversibly bound to the hydrophilic alumina, and thus cannot be separated from the alumina.

U.S. Pat. No. 6,383,783 discloses a method of isolating nucleic acid from a sample, the method including: employing a sample containing target nucleic acids on a hydrophobic organic polymer solid-phase in order to attach target nucleic acid on a solid-phase; and adding a non-ionic surfactant to the solid-phase and removing the attached target nucleic acid. The invention disclosed in U.S. Pat. No. 6,383,783 uses a hydrophilic solid-phase.

### BRIEF SUMMARY OF THE INVENTION

The present invention provides, in an embodiment, a method of purifying nucleic acid using a solid support having a hydrophilic surface, the method including: contacting a nucleic acid-containing sample and a solution containing a kosmotropic (i.e., water-structuring) salt on a solid support having a hydrophilic functional group on its surface to bind the nucleic acid to the solid support.

In another embodiment, the present invention also provides an apparatus for purifying nucleic acid, the apparatus including: a solid support having a hydrophilic functional group on its surface; and a kosmotropic salt solution storing part that is interconnected to the solid support through a microchannel, and provides the kosmotropic salt to the solid support.

In another embodiment, the present invention also provides a lab-on-a-chip including the apparatus for purifying nucleic acid.

### BRIEF DESCRIPTION OF THE DRAWINGS

The above and other features and advantages of the present invention will become more apparent by describing in detail exemplary embodiments thereof with reference to the attached drawings in which:

FIG. 1 is a schematic view showing that nucleic acid is bound to a hydrophilic surface of a solid support in the presence of a kosmotropic salt;

FIG. 2 is a graph showing the binding efficiency of E. coli gDNA that is bound to a silica substrate according to pH;

FIG. 3 is a graph showing the binding efficiency of E. coli gDNA according to a pH and concentration of a kosmotropic salt and chaotropic salt;

FIG. 4 is a graph showing the binding efficiency of E. coli gDNA according to types of a substrate surface; and

FIG. 5 is a graph showing the binding and eluting efficiency of E. coli gDNA using a kosmotropic salt.

### DETAILED DESCRIPTION OF THE INVENTION

The present invention will now be described more fully with reference to the accompanying drawings, in which exemplary embodiments of the invention are shown.

It will be understood that when an element is referred to as being “on” another element, it can be directly on the other element or intervening elements may be present therebetween. In contrast, when an element is referred to as being “disposed on” another element, the elements are understood to be in at least partial contact with each other, unless otherwise specified.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Surprisingly, it has been found that when kosmotropic salts are added onto a solid support having a hydrophilic functional group on its surface, nucleic acids can be bound thereto regardless of pH.

Thus, the present invention provides a method of purifying nucleic acid using a solid support which has a hydrophilic functional group on its surface, the method including: contacting a nucleic acid-containing sample and a solution comprising a kosmotropic salt on the solid support to bind the nucleic acid to the solid support.

In the method, nucleic acids are bound to a solid support surface having a hydrophilic functional group regardless of pH using a kosmotropic salt solution, and a low salt solution is added thereto to elute nucleic acids. In a conventional method of isolating nucleic acid a chaotropic salt should be used, pH should be adjusted, a solid support surface should be modified, or extra specific additives such as polyethylene glycol (“PEG”) should be added. In the method according to an embodiment, there is little limitation on surface conditions of a solid support, and nucleic acids can be bound to a solid support at a wide pH range without specific additives.

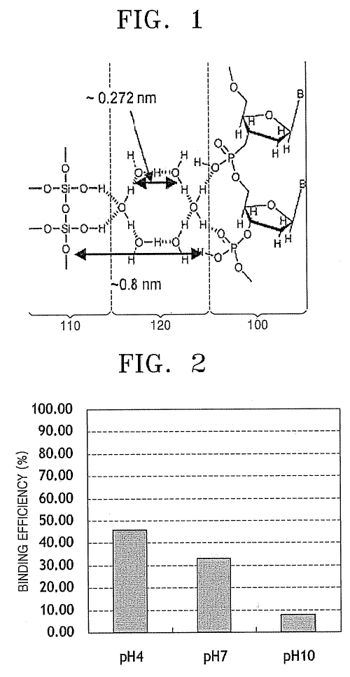

According to an embodiment, a kosmotropic salt is used to bind nucleic acids to a solid support. In the case of a chaotropic salt, a solid support surface is dehydrated and nucleic acids are directly bound to the solid support by hydrogen bonding, so that the pH of the solid support is important. However, in the case of a kosmotropic salt, the solid support surface is hydrated so that a stable water layer is formed thereon. Nucleic acid is considered to be hydrated on the hydrated solid support surface, and it stems from a salting-out effect by hydrophilic interaction. FIG. 1 is a schematic view showing that nucleic acid 100 is bound to the hydrophilic surface of a solid support in the presence of a kosmotropic salt. Referring to FIG. 1, water forms hydrogen bonds on a silica substrate 110 (on the left side of FIG. 1) due to a salting out effect of the kosmotropic salt, and the formed water layer 120 (in the middle portion of FIG. 1) forms hydrogen bonds with nucleic acid 100 (on the right side of FIG. 1) again, so that nucleic acid 100 is bound to the solid support 110 by means of the stable water layer 120.

Therefore, nucleic acid binding with a solid support does not require acidic condition that are generally used for nucleic acid binding, and the solid support surface need not be limited to silica.

Exemplary a hydrophilic functional groups present on a solid support include a hydroxyl group, an amine group, a carboxyl group, a polycarboxyl group, and the like, but are not limited thereto.

The method further comprises washing a sample that is not bound to the solid support after the nucleic acid binding. Through this process, nucleic acid is bound to the solid support, and then a sample that is not bound to the solid support is washed, so that nucleic acid can be purified to provide it in a purer form. A solution used in nucleic acid binding with the solid support can be used as a washing solution.

The method, according to an embodiment, further comprises eluting the nucleic acid bound to the solid support by adding water or a nucleic acid eluting buffer solution. Nucleic acid itself that is bound to the solid support can be used, but eluting the nucleic acid bound to the solid support away from the support is required for the efficient use of the bound nucleic acid, for example, amplification or detection of isolated nucleic acid.

In an embodiment, the kosmotropic salt can be sulfate (SO₄²⁻), phosphate (HPO₄²⁻, hydroxide (OH⁻), fluoride (F⁻), formate (HCOO⁻), acetate (CH₃COO⁻), or the like, but is not limited thereto. The kosmotropic salt induces crystallization of proteins, functions as a salting-out ion for hydrophobic particles, and forms a water structure, according to the Hofmeister series.

In the method according to an embodiment, the nucleic acid-containing sample and a solution containing a kosmotropic salt may have a pH of 3-10. When the pH of the nucleic acid-containing sample and a solution containing a kosmotropic salt is beyond this range, DNA can be physically and chemically denatured, and thus subsequent processes can be affected.

In the method according to ant embodiment, the concentration of the kosmotropic salt may be 100-2,000 mM. When the concentration of the kosmotropic salt is less than 100 mM, the binding efficiency of nucleic acid bound to the solid support decreases. When the concentration of the kosmotropic salt is greater than 2,000 mM, it is difficult to prepare the solution.

In the method according to an embodiment, the solid support can be slide glass, a silicon wafer, a magnetic bead, a polystyrene substrate, a membrane, a metal plate, or the like, but is not limited thereto. The solid support can be any material having a hydrophilic functional group on its surface, but should be insoluble in water. A solid support that is soluble in water is difficult to separate from the nucleic acid solution after the nucleic acid is purified. In addition, use of a solid support having a large surface area is desirable because more hydrophilic functional groups can be included on its surface. Therefore, in the case of a planar solid support, such as glass or a wafer, the surface of the planar solid support is processed to have a pillar structure to increase its surface area.

In an embodiment, the nucleic acid eluting buffer can be phosphate, tris(hydroxymethyl)methane (“Tris”), N-2-hydroxyethylpiperidine-N′-2-ethanesulfonic acid (“HEPES”), 2-N-(cyclohexylamino) ethanesulfonic acid (“CHES”), borate, or the like, but is not limited thereto.

In the method according to an embodiment, the nucleic acid eluting buffer may have a pH of 5-10. When the pH of the nucleic acid eluting buffer is less than pH 5, eluting efficiency of the nucleic acid bound to a solid support decreases. When the pH of the nucleic acid eluting buffer is greater than pH 10, subsequent processes may be affected.

In the method according to an embodiment, the concentration of the nucleic acid eluting buffer may be less than or equal to 100 mM. When the concentration of the nucleic acid eluting buffer is greater than 100 mM, eluting efficiency of the nucleic acid bound to a solid support decreases and also subsequent processes may be affected. If the nucleic acid eluting buffer is water, the concentration of the nucleic acid eluting buffer is 0 mM.

In the method according to an embodiment, the binding of the nucleic acid to the solid support or the eluting of the nucleic acid from the solid support can be performed in a static or fluidic state. Contacting of the nucleic acid and the solid support can be performed both in a static state and in a fluidic state. That is, the solid support is contacted with the nucleic acid while a solution containing the nucleic acid flows in a flow control system. In the flow control system, the solid support can be planar. However, in order to contact more nucleic acid with the solid support, the solid support can have pillar structures.

In the method according to an embodiment, the sample containing the nucleic acid can be blood, serum, urine, saliva, ocular lens fluid, cerebrospinal fluid, milk, ascites fluid, synovial fluid, peritoneal cavity liquid, amniotic fluid, tissue, fermentation broth, cell culture fluid, nucleic acid amplification reaction product, nucleic acid synthesis product, or the like, but is not limited thereto. The sample containing the nucleic acid according to an embodiment may be from a mammal, a plant, bacteria, or yeast. The sample can be in a single cell form or tissue form, and the cell or tissue may stem from cultures in vitro.

The nucleic acid purified using the method according to an embodiment may have any molecular weight, and have a single-stranded form, a double-stranded form, a circular form, a plasmid form, or the like. For example, nucleic acids, such as small oligonucleotides or a nucleic acid molecule of a nucleotide having a length of about 10-50, a longer molecule of a nucleotide having a length of about 1,000-10,000, a nucleic acid having even a larger molecular weight of about 50-500 can be isolated using the method according to an embodiment. Unless otherwise noted, molecular weights of nucleotides as used herein are expressed in thousands of-base pairs (i.e., kilobase pairs, kb).

In the method according to an embodiment, the method further comprises detecting the nucleic acid eluted after eluting nucleic acid from the solid support. The eluted nucleic acid can be detected by electrophoresis, sequencing, or the like.

According to an embodiment, the method further comprises amplifying the nucleic acid eluted after eluting nucleic acid from the solid support. When the nucleic acid is present in a very small amount after elution and cannot be directly detected, the amount of nucleic acid can be amplified using a polymerase chain reaction (“PCR”) method. The amplified nucleic acid can then be easily detected using the above detection methods.

In the method according to an embodiment, amplifying the nucleic acid can be performed without removing a nucleic acid eluting buffer. The nucleic acid eluting buffer has almost the same composition as the buffer used in nucleic acid amplification, so that the nucleic acid eluted in the nucleic acid eluting buffer can be immediately amplified without removing the nucleic acid eluting buffer.

The present invention also provides, in another embodiment, an apparatus for purifying nucleic acid which includes: a solid support having a hydrophilic functional group on its surface; and a kosmotropic salt solution storing part that is interconnected to the solid support through a microchannel and provides a kosmotropic salt to the solid support.

The apparatus for purifying nucleic acid according to an embodiment essentially comprises a kosmotropic salt solution storing part and a solid support having a hydrophilic functional group on its surface. The kosmotropic salt solution storing part is a part that provides the kosmotropic salt to the solid support, and is interconnected to the solid support through a microchannel. When a sample containing nucleic acid to be isolated is introduced into the apparatus, the kosmotropic salt in the kosmotropic salt solution storing part is provided to the solid support, the sample containing nucleic acid and kosmotropic salt are mixed in the solid support, and then the nucleic acid is bound to the solid support by the salting-out effect of the kosmotropic salt. To elute the bound nucleic acid, the apparatus for purifying nucleic acid according to an embodiment further comprises a nucleic acid eluting buffer storing part that is interconnected to the solid support through a microchannel and provides a nucleic acid eluting buffer to the solid support.

In the apparatus according to an embodiment, the solid support can have a planar structure, a pillar structure, a bead structure, a sieve structure, or a combination of structures comprising at least one of the foregoing structures, but is not limited thereto.

In the apparatus according to an embodiment, the solid support can be slide glass, a silicon wafer, a magnetic bead, a polystyrene substrate, a membrane, a metal plate or the like, but is not limited thereto. The solid support can be any material having a hydrophilic functional group on its surface, but should be insoluble in water. A solid support that is soluble in water is difficult to separate from a nucleic acid solution after the nucleic acid is purified. In addition, use of a solid support having a large surface area is desirable because more hydrophilic functional groups can be included on its surface. Therefore, in the case of a planar solid support, such as glass or a wafer, the surface of the planar solid support is processed to have a pillar structure to increase its surface area.

In the apparatus according to an embodiment, the kosmotropic salt that is stored in a kosmotropic salt solution storing part and introduced to a solid support through a micro channel can be sulfate (SO₄²⁻), phosphate (HPO₄²⁻), hydroxide (OH⁻), fluoride (F⁻), formate (HCOO⁻), acetate (CH₃COO⁻), or the like, but is not limited thereto.

In the apparatus according to an embodiment, the apparatus further comprises a nucleic acid amplification part or detection part that can amplify and/or detect the nucleic acid eluted after eluting nucleic acid from the solid support. In the nucleic acid amplification part, if the nucleic acid cannot be directly detected after elution due to its being present in a very small amount, the eluted nucleic acid can be amplified using a PCR device. In the nucleic acid detection part, an electrophoresis device, a sequencing device, or the like can be used in order to see whether an eluted nucleic acid is present.

According to another embodiment, there is also provided a lab-on-a-chip including the apparatus for purifying nucleic acid. In the apparatus for purifying nucleic acid according to an embodiment, each functional element can be implemented by a process-on-a-chip using a known microfluidic technique and a micro electromechanical system (“MEMS”) device, and can be further implemented by a lab-on-a-chip.

Hereinafter, the present invention will be described in further detail with reference to the following examples. These examples are for illustrative purposes only and are not intended to limit the scope.

### COMPARATIVE EXAMPLE 1

### Binding Efficiency of Nucleic Acid According to pH using a Qiagen DNA Purification System

Binding efficiency of nucleic acid according to pH using a Qiagen DNA purification system was determined. Using E. coli lysate including E. coli BL21 gDNA 4,688 ng as a sample including nucleic acid and using a Qiagen kit(Cat. #51306) having a silica surface as a substrate surface, the binding efficiency of nucleic acid was measured at pH 4, 7 and 10.

FIG. 2 is a graph showing binding efficiency of E. coli gDNA that is bound to the silica substrate according to pH. As can be seen in FIG. 2, the nucleic acid binding efficiency is highest at pH 4, and the nucleic acid binding efficiency is lowest at pH 10. Therefore, it can be seen that binding efficiency of nucleic acid is significantly reduced as pH increases. This is because when a pH of a solution increases, negative charges on the substrate surface increase, and an electrostatic repulsive force between the increased negative charges and DNA having negative charges increases, so that DNA binding efficiency decreases.

Therefore, nucleic acid binding is possible at a low pH in the Qiagen DNA purification system that does not use a kosmotropic salt, but nucleic acid binding efficiency is significantly reduced at a high pH, and thus it can be seen that nucleic acid is highly affected by pH while isolating nucleic acid.

### EXAMPLE 1

### Nucleic Acid Binding Efficiency using the Method According to an Embodiment

Binding efficiency of nucleic acid using the method according to an embodiment was determined. The experiment was performed in the same manner as in Comparative Example 1, except that SO₄²⁻ was used as a kosmotropic salt, SCN⁻ was used as a chaotropic salt, and 0, 10, 1,000, and 2,000 mM of SO₄²⁻ and SCN⁻, respectively were used, and nucleic acid binding was performed at pH 4, 6.5-7.5, and 10.

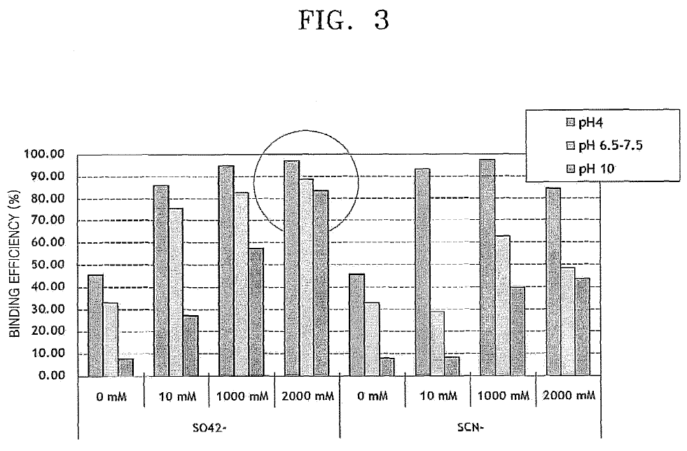

FIG. 3 is a graph showing binding efficiency of E. coli gDNA according to pH and concentration of a kosmotropic salt or chaotropic salt. In FIG. 3, the left panel represents results of measuring binding efficiency of E. coli gDNA using SO₄²⁻ as a kosmotropic salt, and the right panel represents results of measuring binding efficiency of E. coli gDNA using SCN⁻ as a chaotropic salt. As can be seen in FIG. 3, generally, the nucleic acid binding efficiency is the highest at pH 4, and the nucleic acid binding efficiency is the lowest at pH 10, and it can be seen that nucleic acid binding efficiency increases as a concentration of the salts increases. However, unlike Comparative Example 1, in the case of using a kosmotropic salt, as the concentration of SO₄²⁻ increases, binding efficiency of nucleic acid increases even at pH 10. Therefore, it can be seen that binding efficiency of nucleic acid has little difference at pH 4 and pH 10 when a concentration of SO₄²⁻ is 2,000 mM. When a chaotropic salt is used in a low concentration, binding efficiency of nucleic acid is similar to that obtained when a kosmotropic salt is used in a low concentration. However, when the concentration of the salt is 2,000 mM, binding efficiency of nucleic acid decreases as pH increases for the chaotropic salt, unlike that seen with a kosmotropic salt.

The results described above stem from a water hydration effect. It is believed that the kosmotropic salt hydrates the substrate surface so that it enhances a water network, and the strong network of water layer on the silica surface plays a critical role when DNA is bound to the surface by hydrogen bonding.

Therefore, when binding nucleic acid to the surface using a kosmotropic salt according to an embodiment, nucleic acid can be efficiently bound regardless of pH by addition of a proper amount of the kosmotropic salt.

### EXAMPLE 2

### Binding Efficiency of Nucleic Acid According to Substrate Surface Types

Binding efficiency of nucleic acid according to different types of a substrate surface was determined. The substrates used included a glass bead, a glass bead having polycarboxyl terminal groups, or a glass bead having carboxyl terminal groups. The experiment was performed in the same manner as in Comparative Example 1, except that 1 M of sodium sulfate (pH 4, pH 7) was used as a kosmotropic salt, and E. coli gDNA 1.515 ng was used as nucleic acid.

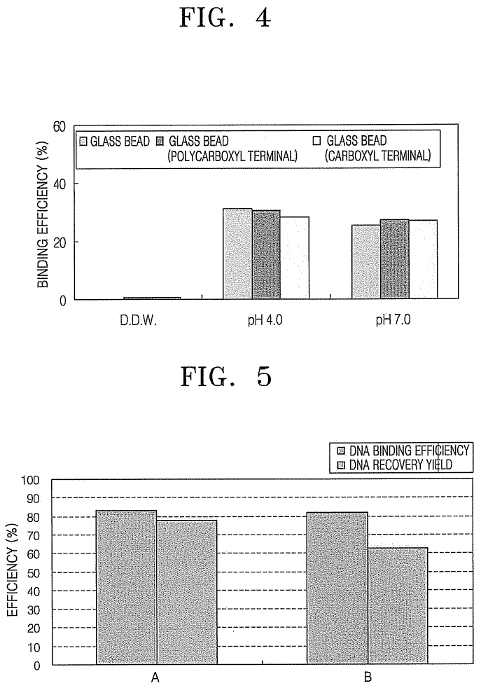

FIG. 4 is a graph showing binding efficiency of E. coli gDNA according to types of a substrate surface. In FIG. 4, results represented by DDW refer to results when distilled water was used in the absence of a kosmotropic salt, and results at pH 4.0 and pH 7.0 refer to results when 1 M of sodium sulfate as a kosmotropic salt was added. As can be seen in FIG. 4, while binding efficiency of nucleic acid is very low when a kosmotropic salt is not added, binding efficiency of nucleic acid is significantly increased when a kosmotropic salt is added. In addition, binding efficiency of nucleic acid at pH 4.0 and pH 7.0 shows little change according to the substrate surface types.

Therefore, when a kosmotropic salt is added while binding nucleic acid to a substrate, nucleic acid is efficiently bound to the substrate regardless of pH or the substrate surface types.

### EXAMPLE 3

### Eluting Efficiency of Nucleic Acid using the Method According to an Embodiment

Eluting efficiency of nucleic acid that was bound to a substrate using the method according to an embodiment was determined. The experiment was performed in the same manner as in Comparative Example 1, except that a silica chip having a pillar structure was used as a substrate, 2M of sodium sulfate (pH 4) was used as a kosmotropic salt, E. coli gDNA 1,377 ng was used as nucleic acid, and 10 mM of Tris-HCl (pH 9) was used as a nucleic acid eluting buffer. The nucleic acid eluting buffer has a similar composition to that of a general PCR buffer. Binding efficiency and eluting efficiency of nucleic acid was compared using a Qiagen solution as a control group.

FIG. 5 is a graph showing binding efficiency and eluting efficiency of E. coli gDNA using a kosmotropic salt. In FIG. 5, A refers to when SO₄²⁻ is used as a kosmotropic salt, and B refers to when a Qiagen solution is used as a control group. As can be seen in FIG. 5, binding efficiency of nucleic acid in both a kosmotropic salt and Qiagen solution is very high at pH 4. However, the DNA recovery yield is higher in the case of using a kosmotropic salt according to an embodiment than in the case of using a Qiagen solution in terms of eluting efficiency.

Therefore, when nucleic acid is purified using a kosmotropic salt, binding efficiency and eluting efficiency of nucleic acid are very high, so that the method according to an embodiment can be efficiently used for nucleic acid purification.

According to the present invention, since a solid support can be used as it is without any surface treatment, manufacture of an apparatus for purifying nucleic acid on hydrophilic surface of a solid support is very easy, and nucleic acid can be bound to the solid support without specific additives in a wide pH range, so that the apparatus can be used for a Lab-On-a-Chip.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A method of purifying nucleic acid using a solid support which has a hydrophilic functional group on its surface, the method comprising:
contacting a nucleic acid-containing sample and a solution comprising a kosmotropic salt on the solid support to bind the nucleic acid to the solid support.

2. The method of claim 1, further comprising eluting the nucleic acid bound to the solid support by addition of water or a nucleic acid eluting buffer.

3. The method of claim 1, wherein the kosmotropic salt is selected from the group consisting of sulfate (SO₄²⁻), phosphate (HPO₄²⁻), hydroxide (OH⁻), fluoride (F⁻), formate (HCOO⁻), and acetate (CH₃COO⁻).

4. The method of claim 1, wherein the nucleic acid-containing sample and the solution containing a kosmotropic salt has a pH of 3 to 10.

5. The method of claim 1 or claim 4, wherein the concentration of the kosmotropic salt is 100 to 2,000 mM.

6. The method of claim 1, wherein the solid support is selected from the group consisting of slide glass, a silicon wafer, a magnetic bead, a polystyrene substrate, a membrane, and a metal plate.

7. The method of claim 2, wherein the nucleic acid eluting buffer is selected from the group consisting of phosphate, Tris, HEPES, CHES, and borate.

8. The method of claim 2, wherein the nucleic acid eluting buffer has a pH of 5 to 10.

9. The method of claim 2, wherein the concentration of the nucleic acid eluting buffer is less than or equal to 100 mM.

10. The method of claim 1, wherein the nucleic acid-containing sample is selected from the group consisting of blood, serum, urine, saliva, ocular lens fluid, cerebrospinal fluid, milk, ascites fluid, synovial fluid, peritoneal cavity liquid, amniotic fluid, tissue, fermentation broth, cell culture fluid, nucleic acid amplification reaction product, and nucleic acid synthesis product.

11. The method of claim 2, further comprising detecting and/or amplifying the eluted nucleic acid after the nucleic acid is eluted from the solid support.

12. The method of claim 11, wherein the amplifying the nucleic acid is performed without removing the nucleic acid eluting buffer.

13. An apparatus for purifying nucleic acid, the apparatus comprising: a solid support having a hydrophilic functional group on its surface; and a kosmotropic salt solution storing part that is interconnected to the solid support through a microchannel, and provides a kosmotropic salt to the solid support.

14. The apparatus of claim 13, further comprising a nucleic acid eluting buffer storing part that is interconnected to the solid support through a microchannel, and provides a nucleic acid eluting buffer to the solid support.

15. The apparatus of claim 13, wherein the solid support has a planar structure, a pillar structure, a bead structure, a sieve structure, or a combination comprising at least one of the foregoing structures.

16. The apparatus of claim 13, wherein the solid support is selected from the group consisting of slide glass, a silicon wafer, a magnetic bead, a polystyrene substrate, a membrane, and a metal plate.

17. The apparatus of claim 13, wherein the kosmotropic salt is selected from the group consisting of sulfate (SO₄²⁻), phosphate (HPO₄²⁻), hydroxide (OH⁻), fluoride (F⁻), formate (HCOO⁻), and acetate (CH₃COO⁻).

18. The apparatus of claim 13, further comprising a nucleic acid amplification part, a nucleic acid detection part, or both a nucleic acid amplification part and a nucleic acid detection part.

19. A lab-on-a-chip comprising the apparatus for purifying nucleic acid of claim 13.
